(19) United States
(12) Patent Application Publication
(10) Pub. No.: US 2021/0148793 A1
(43) Pub. Date: May 20, 2021

(54) SYSTEM AND METHOD FOR MEASURING CORROSION LEVELS IN AIR STREAMS

(71) Applicant: Clear Edge Filtration, Inc., Tulsa, OK (US)

(72) Inventor: Donald Dong Ho Cho, Carrollton, TX (US)

(21) Appl. No.: 17/096,420

(22) Filed: Nov. 12, 2020

Related U.S. Application Data
(60) Provisional application No. 62/935,986, filed on Nov. 15, 2019.

(51) Int. Cl. G01N 1/22

(52) U.S. Cl. CPC G01N 1/2273; G01N 1/2202

(57) ABSTRACT

A system is provided that includes a collection tube extending along a length and having a channel therewithin, wherein one end of the collection tube is configured to receive an air sample. The system further includes a sensor, wherein the sensor is configured to measure a level of corrosion in the air sample in real time. The system also includes a pump, wherein the sensor and pump are fluidly connected in series via the collection tube, wherein the pump is configured to pump the air sample from the one end of the collection tube and through the sensor and the pump.

### RELATED APPLICATIONS AND INCORPORATION BY REFERENCE

This application claims benefit of and priority to U.S. provisional patent application Ser. No. 62/935,986 filed Nov. 15, 2019. The foregoing application, and all documents cited therein or during its prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE DISCLOSURE

The present disclosure generally relates to collection of air samples from high speed air streams, such as from gas turbines, to monitor corrosion and other levels in the air stream in real time.

### BACKGROUND OF THE INVENTION

Heightened corrosion levels in gas turbines can damage the turbines, resulting in increased downtime and maintenance costs. Early detection of heightened corrosion levels can help reduce or eliminate damage, thereby decreasing downtime and maintenance cost. However, for such early detection to be effective, samples from the high speed air stream within the gas turbine must be collected and analyzed in real time (or almost real time). Unfortunately, measuring corrosion causing events from the ambient air stream in gas turbine systems in real time is impractical and unreliable today.

Rather, the prior art consists of systems capable of taking highly sensitive readings that indicate levels of contaminants in the parts per million or even billion. Due to the highly sensitive nature of these systems, they are not designed to be online for long durations of time and are only used under sampling conditions. Thus, the prior art devices are mainly used in laboratory environments or for short field trials to read isolated batches of air samples at a time.

Thus, there exists a need for a system that is capable of continuously measuring the corrosion levels of a high speed air stream in real time.

### BRIEF SUMMARY OF THE INVENTION

In one aspect of the present disclosure, a system is provided. The system comprises a collection tube extending along a length and having a channel therewithin, wherein one end of the collection tube is configured to receive an air sample. The system further comprises a sensor, wherein the sensor is configured to measure a level of corrosion in the air sample in real time. The system also comprises a pump, wherein the sensor and pump are fluidly connected in series via the collection tube, wherein the pump is configured to pump the air sample from the one end of the collection tube and through the sensor and the pump.

In another aspect of the present disclosure, the system may also include a control panel configured to control operation of the sensor and the pump, wherein the control panel is further configured to receive the measure of a level of corrosion in the air sample and transmit the measurements to a user interface. In another aspect of the present disclosure, the collection tube may be a first collection tube, the sensor may be a first sensor, the pump may be a first pump, and the air sample may be a first air sample, and the system may further comprise a second collection tube extending along a length and having a channel therewithin, wherein one end of the second collection tube is configured to receive a second air sample, a second sensor, wherein the second sensor is configured to measure a level of corrosion in a second air sample in real time, and a second pump, wherein the second sensor and second pump are fluidly connected in series via the second collection tube, wherein the second pump is configured to pump the second air sample from the one end of the second collection tube and through the second sensor and the second pump. In yet another aspect, the air sample may be taken from a high speed air stream.

In another aspect of the present disclosure, a method is provided. The method comprises pumping an air sample through a collection tube and through a sensor, wherein the sensor is configured to is configured to measure a level of corrosion in the air sample in real time, wherein the air sample is taken from a high speed air stream.

### BRIEF DESCRIPTION OF THE DRAWINGS

The disclosure will be more readily understood in view of the following description when accompanied by the below figures and wherein like reference numerals represent the elements, wherein:

FIG. 1 is a schematic of one embodiment of a system configured to provide real time measurements of corrosion levels in a high speed air stream.

FIG. 2 is an image of one embodiment of a system configured to provide real time measurements of corrosion levels in a high speed air stream

### DETAILED DESCRIPTION OF THE INVENTION

The following disclosure as a whole may be best understood by reference to the provided detailed description when read in conjunction with the accompanying drawings, drawing description, abstract, background, field of the disclosure, and associated headings. Identical reference numerals when found on different figures identify the same elements or a functionally equivalent element. The elements listed in the abstract are not referenced but nevertheless refer by association to the elements of the detailed description and associated disclosure.

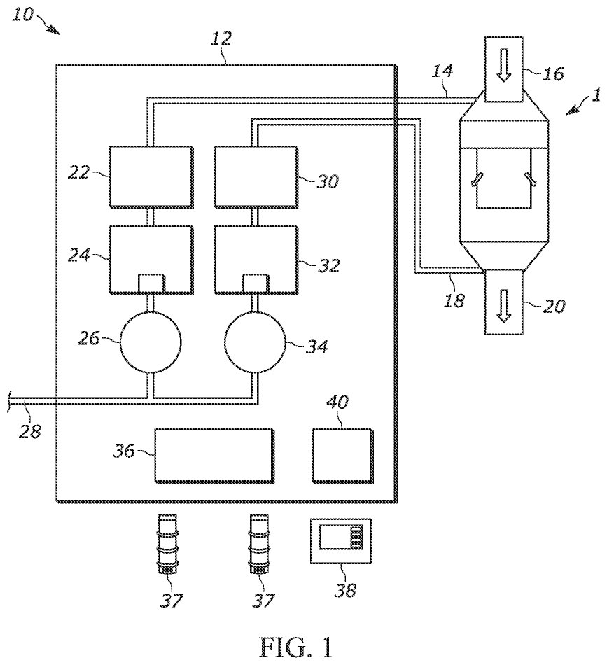

FIG. 1 shows one embodiment of a system 10 configured to monitor corrosion levels of a high speed air stream in real time, such as an air stream from an air filter 1 in a gas turbine. The system 10 may be housed within a cabinet 12 or any other housing. The system 10 may have an inlet collection tube 14 that is fluidly connected to and collects air from an inlet side 16 of the air filter 1 and an outlet collection tube 18 that is fluidly connected to and collects air from an outlet side 20 of the air filter 1. The inlet collection tube 14 and outlet collection tube 18 provide a pathway through which the air samples travel through the system 10. The pathway of the inlet collection tube 14 is as follows: the inlet collection tube 14 may travel from the inlet side 16 of the air filter 1 through an inlet mass flow meter 22 and then on to a first sensor 24. From there, the inlet collection tube 14 travels through an inlet pump 26 and then out into ambient air through an exit port 28. The pathway of the outlet collection tube 18 is similar and is as follows: the outlet collection tube 18 may travel from the outlet side 20 of the air filter 1 through an outlet mass flow meter 30 and then on to a first sensor 32. From there, the outlet collection tube 18 travels through an outlet pump 34 and then out into ambient air through an exit port 28.

Each of the mass flow meters 22, 30, sensors 24, 32, and pumps 28, 34 are electrically connected to a control panel 36, which is configured to control the system 10. The control panel 36 may also be electrically connected to one or more warning lights 37 and a user interface 38, such as a touchscreen. The control panel 36 may also be connected to an IoT gateway 40 or other device that enables device-to-device and device-to-cloud communications. The IoT gateway 40 makes the system 10 internet capable and thus allows it to provide early corrosion warnings to operators of gas turbines at any location and not necessarily on site with the gas turbine.

In operation, the system 10 may monitor the levels of corrosion in the high speed air stream traveling through the filter 1 (or any other air stream, as desired). A user may turn the system 10 on/off and otherwise adjust settings using the user interface 38. Once the system 10 has been turned on, the control panel 36 may direct the inlet and outlet pumps 28, 34 to turn on and start pumping air from the inlet side 16 and outlet side 20 through the inlet tube 14 and outlet tube 18, respectively. The inlet pump 26 thus pumps air from the inlet side 16 of the air filter 1, through the inlet tube 14 and the inlet mass flow meter 22, inlet sensor 24, inlet pump 26, and then out through the exit port 28 into ambient air. Similarly, the outlet pump 34 thus pumps air from the outlet side 20 of the air filter 1, through the outlet tube 18 and the outlet mass flow meter 30, outlet sensor 32, outlet pump 34, and then out through the exit port 28 into ambient air. Alternatively to the exit port 28, the air streams that pass through the inlet and outlet tubes 14, 18 may instead be reintroduced into the regular air stream of the air filter 1 or gas turbine. The sensors 24, 32 may take real time measurements of the corrosion levels of the inlet and outlet air samples, respectively, as they pass through the sensors 24, 32.

Similarly, the mass flow meters 22, 30 may take real time measurements of, and regulate, the mass flow rate of the inlet and outlet air samples, respectively, as they pass through the mass flow meters 22, 30. The mass flow meters 22, 30 assist in regulating the high speed air streams that travel through the system 10, such as by reducing the volume and speed at which the air streams enter the sensors 24, 32. In one embodiment, the mass flow meters 22, 30 may each limit the flow of the air streams into the sensors 24, 32 at a rate of 1 liter of air per minute and cycles twice before each reading. This regulation ensures that the sensors 24, 32 can take accurate readings of the corrosion levels in the air streams.

The control panel 36 may receive these measurements, or inputs, and process and compile them into a useful format for a user of the system 10. In one embodiment, the sensors 24, 32 include two quartz crystal microbalance sensors, one plated in copper and one plated in silver; however, the disclosure is not so limited and other sensors may be used. Each of these crystal microbalance sensors are used to measure the corrosive film that results from the environment. The sensors 24, 32 may transmit the inputs received from the respective crystal microbalance sensors to the control panel 36. The control panel 36 may then store these sensor inputs for a given period of time in a data base. This data base in then used to calculate any increase or decrease in Angstroms in the defined period of time (such as a minute, hour, or day). For example, if the copper value read by the sensors 24, 32 increases from 100 Angstroms to 102 Angstroms in 1 hour, then the increase was 2 Angstroms over 1 hour.

ISA standards require waiting at least 30 days to present a reading on a corrosion level, so instead the control panel 36 may use this data to transmit an expected ISA Class, which may be displayed on the user interface 38 or transmitted elsewhere via the IoT gateway 40. The system 10 is configured to provide G1 to GX corrosion readings according to ISA standards, and these readings can be read over the short, mid, or long term. In one embodiment, the below table demonstrates the corresponding measurement for each ISA Class on corrosion levels.

| | ISA STANDARD S71.04- | SENSORS 24, 32 |
|---|---|---|
| | 1985 | CORRELATION |
| Class G1 | <300 Angstrom over 30 | <10 Angstrom over 24 |
| | days | hours |
| Class G2 | <1000 Angstrom over 30 | <33 Angstrom over 24 |
| | days | hours |
| Class G3 | <2000 Angstrom over 30 | <66 Angstrom over 24 |
| | days | hours |
| Glass GX | >2000 Angstrom over 30 | >67 Angstrom over 24 |
| | days | hours |

For the above example, where an increase of 2 Angstroms over a 1 hour period was detected, this data can be extrapolated to determine an estimated reading over 24 hours, which makes this measurement equivalent to a reading of 48 Angstroms over a 24 hours period. Thus, the corrosion levels in this example are equivalent to Class G3. These levels may be modified as desired, including in a manner that does not correspond to ISA standards, if desired.

The user interface 38 may display multiple measurements over different time periods, which allows a user to see how the measurements are developing over time and whether there are any trends. For example, multiple measurements may indicate that the corrosion levels are rapidly increasing and thus maintenance should be scheduled soon.

For example, the control panel 36 may output readings on the mass flow rate and the corrosion levels of the inlet and outlet air samples to the user interface 38. Alternatively or in addition, the control panel 36 may direct the warning lights 37 to light up in different colors depending on the received measurements, such as the level of corrosion. Red may indicate an urgently high level of corrosion elements whereas green or yellow may indicate a less urgent issue. In addition, the corrosion levels before and after the air travels through the filter 1 may be compared to indicate the effectiveness of the filter 1 and may assist a user of the system 10 in determining when the filter 1 needs to be replaced, cleaned, or otherwise requires maintenance.

While this embodiment describes the mass flow meters 22, 30, sensors 24, 32, and pumps 26, 34 in a particular series and order, the disclosure is not so limited. These components may be rearranged as desired, so long as the sample air stream flows through all components. In addition, this embodiment describes taking samples of the air stream at two separate points (before and after the filter 1); however this disclosure is not so limited. The system 10 may also be configured to take samples at a single point or three or more points, as desired.

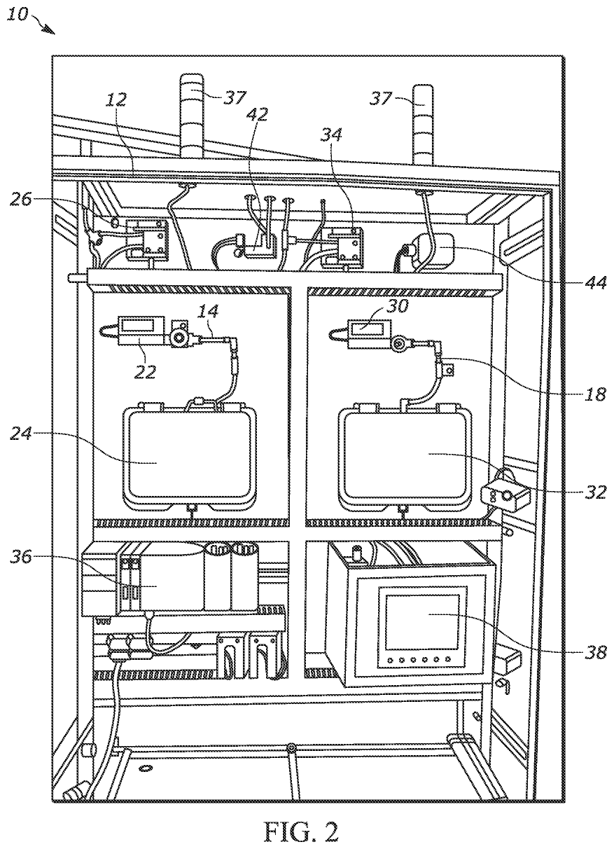

FIG. 2 shows another embodiment of a system 10 that is similar to the embodiment depicted in FIG. 1. As such, the same reference numbers used to describe components with respect to FIG. 1 will be used to reference similar components depicted in the FIG. 2 embodiment. One key difference in this embodiment is the position of the pumps 26, 34. Here, the pumps 26, 34 are positioned in series before the mass flow meters 22, 30 and the sensors 24, 32. In addition, the present embodiment also includes a differential pressure sensor 42 and a temperature sensor 44 that is electrically connected to the control panel 36. The differential pressure sensor 42 may be fluidly connected to both the outlet and inlet air streams travelling through the outlet and inlet tubes 18, 14 respectively and may be configured to measure the pressure difference between these two air streams. This may help indicate whether the air filter 1 is in need of replacing. The temperature sensor 44 may be used to monitor the temperature within the cabinet 12 so as to ensure that the system 10 is not overheating. Alternatively or in addition, the temperature sensor 44, or multiple temperature sensors 44, may be used to measure the air temperature of the air streams traveling through the system 10.

While the above embodiments of the system 10 are described in connection with an air filter 1 for a gas turbine, the disclosure is not so limited. The system 10 can be used to provide real time corrosion data on any high speed air stream, as desired. For example, the system 10 may be used in the oil and gas industries, for sensitive asset protection in long term storage, for corrosion sensitive asset protection, and as an early warning system for transport in ocean or corrosion known areas or shipping.

The above detailed description and the examples described therein have been presented for the purposes of illustration and description only and not by limitation. It is therefore contemplated that the present disclosure cover any and all modifications, variations or equivalents that fall within the spirit and scope of the basic underlying principles disclosed above and claimed herein.

1. A system comprising:
a collection tube extending along a length and having a channel therewithin, wherein one end of the collection tube is configured to receive an air sample;
a sensor, wherein the sensor is configured to measure a level of corrosion in the air sample in real time; and
a pump, wherein the sensor and pump are fluidly connected in series via the collection tube, wherein the pump is configured to pump the air sample from the one end of the collection tube and through the sensor and the pump.

2. The system of claim 1, wherein:
the collection tube is a first collection tube, the sensor is a first sensor, the pump is a first pump, and the air sample is a first air sample,
the system further comprising a second collection tube extending along a length and having a channel therewithin, wherein one end of the second collection tube is configured to receive a second air sample, a second sensor, wherein the second sensor is configured to measure a level of corrosion in a second air sample in real time, and a second pump, wherein the second sensor and second pump are fluidly connected in series via the second collection tube, wherein the second pump is configured to pump the second air sample from the one end of the second collection tube and through the second sensor and the second pump.

3. The system of claim 2, wherein the first and second air samples are taken from a high speed air stream.

4. The system of claim 2, further comprising:
a control panel configured to control operation of the first and second sensors and the first and second pumps, wherein the control panel is further configured to receive the measure of a level of corrosion in the first and second air samples and transmit the measurements to a user interface.

5. The system of claim 2, further comprising at least one light configured to display different colors to indicate the measure of a level of corrosion in the first and second air samples.

6. The system of claim 1, wherein the air sample is taken from a high speed air stream.

7. The system of claim 1, further comprising:
a control panel configured to control operation of the sensor and the pump, wherein the control panel is further configured to receive the measure of a level of corrosion in the air sample and transmit the measurements to a user interface.

8. The system of claim 1, further comprising a light configured to display different colors to indicate the measure of a level of corrosion in the air sample.

9. The system of claim 1, further comprising a mass flow meter connected in series with the pump and the sensor via the collection tube, wherein the mass flow meter is configured to regulate the rate at which the air enters the collection tube.

10. The system of claim 1, further comprising a temperature sensor configured to detect the temperature of the air flowing through the collection tube.

11. The system of claim 9, further comprising a control panel configured to control operation of the sensor, the pump, and the mass flow meter, wherein the control panel is further configured to receive the measure of a level of corrosion in the air sample and transmit the measurements to a user interface.

12. The system of claim 10, further comprising a control panel configured to control operation of the sensor, the pump, and the temperature sensor, wherein the control panel is further configured to receive the measure of a level of corrosion in the air sample and transmit the measurements to a user interface.

13. The system of claim 1, wherein the sensor comprises a quartz crystal microbalance sensor.

14. The system of claim 2, wherein the first and second sensors each comprise a quartz crystal microbalance sensor.

15. The system of claim 14, wherein the first sensor is plated in copper and the second sensor is plated in silver.

16. A method comprising:
pumping an air sample through a collection tube and through a sensor, wherein the sensor is configured to measure a level of corrosion in the air sample in real time, wherein the air sample is taken from a high speed air stream.

17. The method of claim 16, wherein the collection tube is a first collection tube, the sensor is a first sensor, the pump is a first pump, and the air sample is a first air sample, the method further comprising the step of:
pumping a second air sample through a second collection tube and through a second sensor, wherein the second sensor is configured to measure a level of corrosion in the second air sample in real time, wherein the second air sample is taken from the high speed air stream.

18. The method of claim 16, wherein the sensor comprises a quartz crystal microbalance sensor.

19. The method of claim 17, wherein the first and second sensors each comprise a quartz crystal microbalance sensor.

20. The method of claim 19, wherein the first sensor is plated in copper and the second sensor is plated in silver.
